(19) United States
(12) Patent Application Publication
(10) Pub. No.: US 2011/0129514 A1
(43) Pub. Date: Jun. 2, 2011

(54) Hygroscopic coating on a balloon device

(76) Inventors: Syed F.A. Hossainy, Fremont, CA (US); Mikael O. Trollsas, San Jose, CA (US); Keith Cromack, (US)

(21) Appl. No.: 11/899,792

(22) Filed: Sep. 6, 2007

(51) Int. Cl. A61K 9/00; A61P 9/00; A61F 2/84

(52) U.S. Cl. 424/423; 623/1.11

(57) ABSTRACT

The present invention provides a hygroscopic coating on a balloon for an implantable device and methods of making and using the same.

### FIELD OF THE INVENTION

The present invention relates to a hygroscopic coating on a balloon device.

### BACKGROUND OF THE INVENTION

Percutaneous coronary intervention (PCI) is a procedure for treating heart disease. A catheter assembly having a balloon portion is introduced percutaneously into the cardiovascular system of a patient via the brachial or femoral artery. The catheter assembly is advanced through the coronary vasculature until the balloon portion is positioned across the occlusive lesion. Once in position across the lesion, the balloon is inflated to a predetermined size to radially compress the atherosclerotic plaque of the lesion to remodel the lumen wall. The balloon is then deflated to a smaller profile to allow the catheter to be withdrawn from the patient's vasculature.

Problems associated with the above procedure include formation of intimal flaps or torn arterial linings which can collapse and occlude the blood conduit after the balloon is deflated. Moreover, thrombosis and restenosis of the artery may develop over several months after the procedure, which may require another angioplasty procedure or a surgical by-pass operation. To reduce the partial or total occlusion of the artery by the collapse of the arterial lining and to reduce the chance of thrombosis or restenosis, a stent is implanted in the artery to keep the artery open.

Drug delivery stents have reduced the incidence of in-stent restenosis (ISR) after PCI (see, e.g., Serruys, P. W., et al., J. Am. Coll. Cardiol. 39:393-399 (2002)), which has plagued interventional cardiology for more than a decade. However, a few challenges remain in the art of drug delivery stents. An example of such challenges is the loss of therapeutic agent in the deployment of the stent.

The embodiments of the present invention address the above-identified needs and issues.

### SUMMARY OF THE INVENTION

The present invention provides a coating on a balloon including a hygroscopic coating. The coating includes a hygroscopic layer comprising a hygroscopic polymer(s). The hygroscopic layer is substantially free from water or moisture and capable of absorbing water (water absorbing capacity) at about 5 wt % or more of the total weight of the hygroscopic layer water. In some embodiments, the hygroscopic layer can have a water absorbing capacity of about 10 wt % or higher, about 20 wt % or higher, about 40 wt % or higher, about 50 wt % or higher, about 75 wt % or higher, about 90 wt % or higher, about 100 wt % or higher, about 200 wt % or higher, about 400 wt % or higher, about 500 wt % or higher, about 600 wt % or higher, about 700 wt % or higher, about 800 wt % or higher, about 900 wt % or higher, or about 1000 wt % or higher, all by the weight of the hygroscopic layer.

In some embodiments, the hygroscopic layer can have a water absorbing capacity with an upper limit of about 20 wt %, about 40 wt %, about 60 wt %, about 80 wt %, about 100 wt %, about 500 wt %, about 600 wt %, about 700 wt %, about 800 wt %, about 900 wt %, or about 1000 wt %.

In some embodiments, the hygroscopic coating described herein can have a fast water absorbing rate. For example, a hygroscopic coating can absorb or swell a percentage of water by the dry weight of the hygroscopic coating within about 1 minute, about 2 minutes, about 5 minutes, about 10 minutes, about 20 minutes, about 30 minutes, or about 60 minutes upon exposure to an aqueous environment. Such percentage of water can be, e.g., about 5 wt %, about 10 wt %, about 20 wt %, about 40 wt %, about 50 wt %, about 75 wt %, about 90 wt %, about 100 wt %, about 200 wt %, about 400 wt %, about 500 wt %, about 600 wt %, about 700 wt %, about 800 wt %, about 900 wt %, or about 1000 wt %, all by the dry weight of the hygroscopic layer.

In some embodiments, the hygroscopic layer is capable of absorbing between about 20 wt % and about 500 wt % by the dry weight of the hygroscopic layer within about 5 to 60 minutes upon exposure to an aqueous environment.

In some embodiments, the hygroscopic layer can be formed on an implantable device such as a drug delivery stent to minimize drug loss into unwanted areas during stent deployment by its water-absorbing capability to cause a drug in the coating not to leak into environment. In these embodiments, the hygroscopic layer can be a topcoat or a non-topcoat layer in a coating on the implantable device.

In some embodiments, the hygroscopic layer can be formed on a balloon of an implantable device such as a drug delivery stent for loading a drug in situ by exposing the hygroscopic coating to an aqueous solution of a drug, forming a drug loaded hydrogel coating in situ so as to minimize drug loss that may result from stent fabrication, packaging or handling. In these embodiments, the hygroscopic layer can be used to optionally load a second drug or agent. For example, the hygroscopic layer can be included in a coating that has a reservoir layer including a drug or agent. The hygroscopic layer therefore can be used to load additional drug or agents, in addition to the drug or agent in the reservoir layer.

In addition to a hygroscopic polymer or material described below, the hygroscopic layer can include one or more non-hygroscopic polymer or material. Such non-hygroscopic polymer can be degradable or non-degradable, which is further described below.

Some exemplary bioactive agents that can be included in a coating having a hygroscopic layer described above are paclitaxel, docetaxel, estradiol, 17-beta-estradiol, nitric oxide donors, super oxide dismutases, super oxide dismutases mimics, 4-amino-2,2,6,6-tetramethylpiperidine-1-oxyl (4-amino-TEMPO), biolimus, tacrolimus, dexamethasone, dexamethasone-acetate, rapamycin, rapamycin derivatives, 40-O-(2-hydroxy)ethyl-rapamycin (everolimus), 40-O-(3-hydroxy)propyl-rapamycin, 40-O-[2-(2-hydroxy)ethoxy]ethyl-rapamycin, and 40-O-tetrazole-rapamycin, 40-epi-(N1-tetrazolyl)-rapamycin (ABT-578), γ-hiridun, clobetasol, pimecrolimus, imatinib mesylate, midostaurin, cRGD, prodrugs thereof, co-drugs thereof, and combinations thereof. Some other examples of the bioactive agent include siRNA and/or other oligonucleotides that inhibit endothelial cell migration. Some further examples of the bioactive agent can also be lysophosphatidic acid (LPA) or sphingosine-1-phosphate (S1P). LPA is a “bioactive” phospholipid able to generate growth factor-like activities in a wide variety of normal and malignant cell types. LPA plays an important role in normal physiological processes such as wound healing, and in vascular tone, vascular integrity, or reproduction.

The hygroscopic layer can be formed on a balloon for an implantable device such as a stent, which can be implanted in a patient to treat, prevent, mitigate, or reduce a vascular medical condition, or to provide a pro-healing effect. In some embodiments, the vascular medical condition or vascular condition is a coronary artery disease (CAD) and/or a peripheral vascular disease (PVD). Some examples of such vascular medical diseases are restenosis and/or atherosclerosis. Some other examples of these conditions include thrombosis, hemorrhage, vascular dissection or perforation, vascular aneurysm, vulnerable plaque, chronic total occlusion, claudication, anastomotic proliferation (for vein and artificial grafts), bile duct obstruction, ureter obstruction, tumor obstruction, or combinations of these.

### DETAILED DESCRIPTION

The present invention provides a balloon including a hygroscopic coating. The coating includes a hygroscopic layer comprising a hygroscopic polymer(s). The hygroscopic layer is substantially free from water or moisture and capable of absorbing water (water absorbing capacity) at about 5 wt % or more of the total weight of the hygroscopic layer water. In some embodiments, the hygroscopic layer can have a water absorbing capacity of about 10 wt % or higher, about 20 wt % or higher, about 40 wt % or higher, about 50 wt % or higher, about 75 wt % or higher, about 90 wt % or higher, about 100 wt % or higher, about 200 wt % or higher, about 400 wt % or higher, about 500 wt % or higher, about 600 wt % or higher, about 700 wt % or higher, about 800 wt % or higher, about 900 wt % or higher, or about 1000 wt % or higher, all by the dry weight of the hygroscopic layer.

In some embodiments, the hygroscopic layer can have a water absorbing capacity with an upper limit of about 20 wt %, about 40 wt %, about 60 wt %, about 80 wt %, about 100 wt %, about 500 wt %, about 600 wt %, about 700 wt %, about 800 wt %, about 900 wt %, or about 1000 wt %.

As used herein, the term “substantially free from water or moisture” shall mean having a water or moisture content less than about 10% wt, less than about 5 wt %, less than about 1 wt %, less than about 0.1 wt %, or less than about 0.01 wt % water.

In some embodiments, the hygroscopic coating described herein can have a fast water absorbing rate. For example, a hygroscopic coating can absorb or swell a percentage of water by the dry weight of the hygroscopic coating within about 1 minute, about 2 minutes, about 5 minutes, about 10 minutes, about 20 minutes, about 30 minutes, or about 60 minutes upon exposure to an aqueous environment. Such percentage of water can be, e.g., about 5 wt %, about 10 wt %, about 20 wt %, about 40 wt %, about 50 wt %, about 75 wt %, about 90 wt %, about 100 wt %, about 200 wt %, about 400 wt %, about 500 wt %, about 600 wt %, about 700 wt %, about 800 wt %, about 900 wt %, or about 1000 wt %, all by the dry weight of the hygroscopic layer.

In some embodiments, the hygroscopic layer is capable of absorbing between about 20 wt % and about 500 wt % by the dry weight of the hygroscopic layer within about 5 to 60 minutes upon exposure to an aqueous environment.

As used herein, the term “absorb” can be used interchangeably with the term “swell.”

In some embodiments, the hygroscopic layer can be formed on a drug coated balloon for an implantable device such as a drug delivery stent to minimize drug loss into unwanted areas during stent deployment by its water-absorbing capability to cause a drug in the coating not to leak into environment. In these embodiments, the hygroscopic layer can be a topcoat or a non-topcoat layer in a coating on the implantable device.

In some embodiments, the hygroscopic layer can be formed on balloon for an implantable device such as a drug delivery stent for loading a drug in situ by exposing the hygroscopic coating to an aqueous solution of a drug, forming a drug loaded hydrogel coating in situ so as to minimize drug loss that may result from stent fabrication, packaging or handling. In these embodiments, the hygroscopic layer can be used to optionally load a second drug or agent. For example, the hygroscopic layer can be included in a coating that has a reservoir layer including a drug or agent. The hygroscopic layer therefore can be used to load additional drug or agents, in addition to the drug or agent in the reservoir layer.

In some embodiments, the hygroscopic layer is frangible such that upon expansion of the balloon the hygroscopic layer breaks so as to allow the agent in the drug layer to quickly release into the aqueous environment. As used herein, the term “quickly release” refers to a release rate of up to about 50% to about 100% of the agent or drug from the drug layer or reservoir layer on the balloon within a period of about 5 minutes to about 60 minutes.

In addition to a hygroscopic polymer or material described below, the hygroscopic layer can include one or more non-hygroscopic polymer or material. Such non-hygroscopic polymer can be degradable or non-degradable, which is further described below.

Some exemplary bioactive agents that can be included in a coating having a hygroscopic layer described above are paclitaxel, docetaxel, estradiol, 17-beta-estradiol, nitric oxide donors, super oxide dismutases, super oxide dismutases mimics, 4-amino-2,2,6,6-tetramethylpiperidine-1-oxyl (4-amino-TEMPO), biolimus, tacrolimus, dexamethasone, dexamethasone-acetate, rapamycin, rapamycin derivatives, 40-O-(2-hydroxy)ethyl-rapamycin (everolimus), 40-O-(3-hydroxy)propyl-rapamycin, 40-O-[2-(2-hydroxy)ethoxy]ethyl-rapamycin, and 40-O-tetrazole-rapamycin, 40-epi-(N1-tetrazolyl)-rapamycin (ABT-578), γ-hiridun, clobetasol, pimecrolimus, imatinib mesylate, midostaurin, cRGD, prodrugs thereof, co-drugs thereof, and combinations thereof. Some other examples of the bioactive agent include siRNA and/or other oligonucleotides that inhibit endothelial cell migration. Some further examples of the bioactive agent can also be lysophosphatidic acid (LPA) or sphingosine-1-phosphate (S1P). LPA is a “bioactive” phospholipid able to generate growth factor-like activities in a wide variety of normal and malignant cell types. LPA plays an important role in normal physiological processes such as wound healing, and in vascular tone, vascular integrity, or reproduction.

The implantable device described herein can be formed on an implantable device such as a stent, which can be implanted in a patient to treat, prevent, mitigate, or reduce a vascular medical condition, or to provide a pro-healing effect. In some embodiments, the vascular medical condition or vascular condition is a coronary artery disease (CAD) and/or a peripheral vascular disease (PVD). Some examples of such vascular medical diseases are restenosis and/or atherosclerosis. Some other examples of these conditions include thrombosis, hemorrhage, vascular dissection or perforation, vascular aneurysm, vulnerable plaque, chronic total occlusion, claudication, anastomotic proliferation (for vein and artificial grafts), bile duct obstruction, ureter obstruction, tumor obstruction, or combinations of these.

### DEFINITIONS

Wherever applicable, the definitions to some terms used throughout the description of the present invention as provided below shall apply.

The terms “biologically degradable” (or “biodegradable”), “biologically erodable” (or “bioerodable”), “biologically absorbable” (or “bioabsorbable”), and “biologically resorbable” (or “bioresorbable”), in reference to polymers and coatings, are used interchangeably and refer to polymers and coatings that are capable of being completely or substantially completely degraded, dissolved, and/or eroded over time when exposed to physiological conditions and can be gradually resorbed, absorbed and/or eliminated by the body, or that can be degraded into fragments that can pass through the kidney membrane of an animal (e.g., a human), e.g., fragments having a molecular weight of about 40,000 Daltons (40 kDa) or less. The process of breaking down and eventual absorption and elimination of the polymer or coating can be caused by, e.g., hydrolysis, metabolic processes, oxidation, enzymatic processes, bulk or surface erosion, and the like. Conversely, a “biostable” polymer or coating refers to a polymer or coating that is not biodegradable.

Whenever the reference is made to “biologically degradable,” “biologically erodable,” “biologically absorbable,” and “biologically resorbable” stent coatings or polymers forming such stent coatings, it is understood that after the process of degradation, erosion, absorption, and/or resorption has been completed or substantially completed, no coating or substantially little coating will remain on the stent. Whenever the terms “degradable,” “biodegradable,” or “biologically degradable” are used in this application, they are intended to broadly include biologically degradable, biologically erodable, biologically absorbable, and biologically resorbable polymers or coatings.

“Physiological conditions” refer to conditions to which an implant is exposed within the body of an animal (e.g., a human). Physiological conditions include, but are not limited to, “normal” body temperature for that species of animal (approximately 37° C. for a human) and an aqueous environment of physiologic ionic strength, pH and enzymes. In some cases, the body temperature of a particular animal may be above or below what would be considered “normal” body temperature for that species of animal. For example, the body temperature of a human may be above or below approximately 37° C. in certain cases. The scope of the present invention encompasses such cases where the physiological conditions (e.g., body temperature) of an animal are not considered “normal.”

In the context of a blood-contacting implantable device, a “prohealing” drug or agent refers to a drug or agent that has the property that it promotes or enhances re-endothelialization of arterial lumen to promote healing of the vascular tissue.

As used herein, a “co-drug” is a drug that is administered concurrently or sequentially with another drug to achieve a particular pharmacological effect. The effect may be general or specific. The co-drug may exert an effect different from that of the other drug, or it may promote, enhance or potentiate the effect of the other drug.

As used herein, the term “prodrug” refers to an agent rendered less active by a chemical or biological moiety, which metabolizes into or undergoes in vivo hydrolysis to form a drug or an active ingredient thereof. The term “prodrug” can be used interchangeably with terms such as “proagent”, “latentiated drugs”, “bioreversible derivatives”, and “congeners”. N. J. Harper, Drug latentiation, Prog Drug Res., 4: 221-294 (1962); E. B. Roche, Design of Biopharmaceutical Properties through Prodrugs and Analogs, Washington, D.C.: American Pharmaceutical Association (1977); A. A. Sinkula and S. H. Yalkowsky, Rationale for design of biologically reversible drug derivatives: prodrugs, J. Pharm. Sci., 64: 181-210 (1975). Use of the term “prodrug” usually implies a covalent link between a drug and a chemical moiety, though some authors also use it to characterize some forms of salts of the active drug molecule. Although there is no strict universal definition of a prodrug itself, and the definition may vary from author to author, prodrugs can generally be defined as pharmacologically less active chemical derivatives that can be converted in vivo, enzymatically or nonenzymatically, to the active, or more active, drug molecules that exert a therapeutic, prophylactic or diagnostic effect. Sinkula and Yalkowsky, above; V. J. Stella et al., Prodrugs: Do they have advantages in clinical practice?, Drugs, 29: 455-473 (1985).

The terms “polymer” and “polymeric” refer to compounds that are the product of a polymerization reaction. These terms are inclusive of homopolymers (i.e., polymers obtained by polymerizing one type of monomer), copolymers (i.e., polymers obtained by polymerizing two or more different types of monomers), terpolymers, etc., including random, alternating, block, graft, dendritic, crosslinked and any other variations thereof.

As used herein, the term “implantable” refers to the attribute of being implantable in a mammal (e.g., a human being or patient) that meets the mechanical, physical, chemical, biological, and pharmacological requirements of a device provided by laws and regulations of a governmental agency (e.g., the U.S. FDA) such that the device is safe and effective for use as indicated by the device. As used herein, an “implantable device” may be any suitable substrate that can be implanted in a human or non-human animal. Examples of implantable devices include, but are not limited to, self-expandable stents, balloon-expandable stents, coronary stents, peripheral stents, stent-grafts, catheters, other expandable tubular devices for various bodily lumen or orifices, grafts, vascular grafts, arterio-venous grafts, by-pass grafts, pacemakers and defibrillators, leads and electrodes for the preceding, artificial heart valves, anastomotic clips, arterial closure devices, patent foramen ovale closure devices, cerebrospinal fluid shunts, and particles (e.g., drug-eluting particles, microparticles and nanoparticles). The stents may be intended for any vessel in the body, including neurological, carotid, vein graft, coronary, aortic, renal, iliac, femoral, popliteal vasculature, and urethral passages. An implantable device can be designed for the localized delivery of a therapeutic agent. A medicated implantable device may be constructed in part, e.g., by coating the device with a coating material containing a therapeutic agent. The body of the device may also contain a therapeutic agent.

An implantable device can be fabricated with a coating containing partially or completely a biodegradable/bioabsorbable/bioerodable polymer, a biostable polymer, or a combination thereof. An implantable device itself can also be fabricated partially or completely from a biodegradable/bioabsorbable/bioerodable polymer, a biostable polymer, or a combination thereof.

As used herein, a material that is described as a layer or a film (e.g., a coating) “disposed over” an indicated substrate (e.g., an implantable device) refers to, e.g., a coating of the material deposited directly or indirectly over at least a portion of the surface of the substrate. Direct depositing means that the coating is applied directly to the exposed surface of the substrate. Indirect depositing means that the coating is applied to an intervening layer that has been deposited directly or indirectly over the substrate. In some embodiments, the term a “layer” or a “film” excludes a film or a layer formed on a non-implantable device.

In the context of a stent, “delivery” refers to introducing and transporting the stent through a bodily lumen to a region, such as a lesion, in a vessel that requires treatment. “Deployment” corresponds to the expanding of the stent within the lumen at the treatment region. Delivery and deployment of a stent are accomplished by positioning the stent about one end of a catheter, inserting the end of the catheter through the skin into a bodily lumen, advancing the catheter in the bodily lumen to a desired treatment location, expanding the stent at the treatment location, and removing the catheter from the lumen.

### Hygroscopic Polymers

Any polymer substantially free from water or moisture and has a water absorbing capacity as defined above can be used to form the hygroscopic layer defined above.

In some embodiments, the polymer is a hygroscopic, hydrophilic polymer. Some examples of hygroscopic, hydrophilic polymers include high molecular weight hydrophilic polymers such as polyvinylpyrrolidone (PVP); poly(carboxymethyl cellulose) (poly(CMC)), and poly[N-(2-hydroxypropyl)methacrylamide] (poly(HPMA)).

In some embodiments, the hygroscopic polymer can be an amphilic polymer. In some further embodiments, the hygroscopic polymer can be a hydrophobic polymer comprising hygroscopic moieties or pendant groups. Such hygroscopic moieties or pendant groups include, but are not limited to, choline, phosphoryl choline, ammonium groups, sulfuric acid groups, and sulfonic acid groups. Some examples of hygroscopic, amphiphilic polymers include, but are not limited to, poly(n-butyl methacrylate-phosphorylcholine) (PBMA-PC), poly(ester amide)-phosphorylcholine (PEA-PC), polylactide-phosphorylcholine (PLA-PC), polylactide-phosphorylcholine (PLA-PC), polyethylene glycol-poly(caprolactone)-di- or tri-blocks (PEG-PCL), polyethylene glycol-polylactide di- or tri-blocks (PEG-PLA), polyethylene glycol-poly(lactide-glycolide) di- or tri-blocks (PEG-PLGA), Pluronic®, etc.

In some further embodiments, the hygroscopic polymer can be a dissolvable polymer. As used herein, the term dissolvable polymer refers to a polymer that can be dissolved or is soluble (e.g., capable of forming a water solution of 1 g/100 mL water at ambient temperature) in an aqueous environment. Some examples of hygroscopic, dissolvable polymers include, but are not limited to, de-acetylated polyvinyl acetate (PVAc); polyvinyl alcohol (PVA); poly n-butyl methacrylate-phosphorylcholine (PBMA-PC); poly(ester amide)-phosphorylcholine (PEA-PC); polylactide-phosphorylcholine (PLA-PC); poly(lactic acid-co-glycolic acid)-phosphorylcholine (PLGA-PC); silk-elastin, dynamers, or tri-blocks such as poly(caprolactone)-polyethylene glycol-poly(caprolactone) (PCL-PEG-PCL); (PLA-PEG-PLA); and poly(lactide-glycolide)-polyethylene glycol-poly(lactide-glycolide) tri-blocks (PLGA-PEG-PLGA).

### Biologically Active Agents

In addition to the ACAT inhibitor described above, the drug layer on the balloon and/or the implantable device described herein can optionally include at least one biologically active (“bioactive”) agent. The at least one bioactive agent can include any substance capable of exerting a therapeutic, prophylactic or diagnostic effect for a patient.

Examples of suitable bioactive agents include, but are not limited to, synthetic inorganic and organic compounds, proteins and peptides, polysaccharides and other sugars, lipids, and DNA and RNA nucleic acid sequences having therapeutic, prophylactic or diagnostic activities. Nucleic acid sequences include genes, antisense molecules that bind to complementary DNA to inhibit transcription, and ribozymes. Some other examples of other bioactive agents include antibodies, receptor ligands, enzymes, adhesion peptides, blood clotting factors, inhibitors or clot dissolving agents such as streptokinase and tissue plasminogen activator, antigens for immunization, hormones and growth factors, oligonucleotides such as antisense oligonucleotides and ribozymes and retroviral vectors for use in gene therapy. The bioactive agents could be designed, e.g., to inhibit the activity of vascular smooth muscle cells. They could be directed at inhibiting abnormal or inappropriate migration and/or proliferation of smooth muscle cells to inhibit restenosis.

In certain embodiments, optionally in combination with one or more other embodiments described herein, the implantable device can include at least one biologically active agent selected from antiproliferative, antineoplastic, antimitotic, anti-inflammatory, antiplatelet, anticoagulant, antifibrin, antithrombin, antibiotic, antiallergic and antioxidant substances.

An antiproliferative agent can be a natural proteineous agent such as a cytotoxin or a synthetic molecule. Examples of antiproliferative substances include, but are not limited to, actinomycin D or derivatives and analogs thereof (manufactured by Sigma-Aldrich, or COSMEGEN available from Merck) (synonyms of actinomycin D include dactinomycin, actinomycin IV, actinomycin I₁, actinomycin X₁, and actinomycin C₁); all taxoids such as taxols, docetaxel, and paclitaxel and derivatives thereof; all olimus drugs such as macrolide antibiotics, rapamycin, everolimus, structural derivatives and functional analogues of rapamycin, structural derivatives and functional analogues of everolimus, FKBP-12 mediated mTOR inhibitors, biolimus, perfenidone, prodrugs thereof, co-drugs thereof, and combinations thereof. Examples of rapamycin derivatives include, but are not limited to, 40-O-(2-hydroxy)ethyl-rapamycin (trade name everolimus from Novartis), 40-O-(2-ethoxy)ethyl-rapamycin (biolimus), 40-O-(3-hydroxy)propyl-rapamycin, 40-O-[2-(2-hydroxy)ethoxy]ethyl-rapamycin, 40-O-tetrazole-rapamycin, 40-epi-(N1-tetrazolyl)-rapamycin (zotarolimus, manufactured by Abbott Labs.), prodrugs thereof, co-drugs thereof, and combinations thereof.

An anti-inflammatory drug can be a steroidal anti-inflammatory drug, a nonsteroidal anti-inflammatory drug (NSAID), or a combination thereof. Examples of anti-inflammatory drugs include, but are not limited to, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol, clobetasol propionate, clobetasone butyrate, clopirac, cloticasone propionate, cormethasone acetate, cortodoxone, deflazacort, desonide, desoximetasone, dexamethasone, dexamethasone acetate, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, momiflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid), salicylic acid, corticosteroids, glucocorticoids, tacrolimus, pimecorlimus, prodrugs thereof, co-drugs thereof, and combinations thereof.

Alternatively, the anti-inflammatory agent can be a biological inhibitor of pro-inflammatory signaling molecules. Anti-inflammatory biological agents include antibodies to such biological inflammatory signaling molecules.

In addition, the bioactive agents can be other than antiproliferative or anti-inflammatory agents. The bioactive agents can be any agent that is a therapeutic, prophylactic or diagnostic agent. In some embodiments, such agents can be used in combination with antiproliferative or anti-inflammatory agents. These bioactive agents can also have antiproliferative and/or anti-inflammatory properties or can have other properties such as antineoplastic, antimitotic, cystostatic, antiplatelet, anticoagulant, antifibrin, antithrombin, antibiotic, antiallergic, and/or antioxidant properties.

Examples of antineoplastics and/or antimitotics include, but are not limited to, paclitaxel (e.g., TAXOL® available from Bristol-Myers Squibb), docetaxel (e.g., Taxotere® from Aventis), methotrexate, azathioprine, vincristine, vinblastine, fluorouracil, doxorubicin hydrochloride (e.g., Adriamycin® from Pfizer), and mitomycin (e.g., Mutamycin® from Bristol-Myers Squibb).

Examples of antiplatelet, anticoagulant, antifibrin, and antithrombin agents that can also have cytostatic or antiproliferative properties include, but are not limited to, sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, thrombin inhibitors such as ANGIOMAX (from Biogen), calcium channel blockers (e.g., nifedipine), colchicine, fibroblast growth factor (FGF) antagonists, fish oil (e.g., omega 3-fatty acid), histamine antagonists, lovastatin (a cholesterol-lowering drug that inhibits HMG-CoA reductase, brand name Mevacor® from Merck), monoclonal antibodies (e.g., those specific for platelet-derived growth factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), nitric oxide or nitric oxide donors, super oxide dismutases, super oxide dismutase mimetics, 4-amino-2,2,6,6-tetramethylpiperidine-1-oxyl (4-amino-TEMPO), estradiol, anticancer agents, dietary supplements such as various vitamins, and a combination thereof.

Examples of cytostatic substances include, but are not limited to, angiopeptin, angiotensin converting enzyme inhibitors such as captopril (e.g., Capoten® and Capozide® from Bristol-Myers Squibb), cilazapril and lisinopril (e.g., Prinivil® and Prinzide® from Merck).

Examples of antiallergic agents include, but are not limited to, permirolast potassium. Examples of antioxidant substances include, but are not limited to, 4-amino-2,2,6,6-tetramethylpiperidine-1-oxyl (4-amino-TEMPO). Other bioactive agents include anti-infectives such as antiviral agents; analgesics and analgesic combinations; anorexics; antihelmintics; antiarthritics, antiasthmatic agents; anticonvulsants; antidepressants; antidiuretic agents; antidiarrheals; antihistamines; antimigrain preparations; antinauseants; antiparkinsonism drugs; antipruritics; antipsychotics; antipyretics; antispasmodics; anticholinergics; sympathomimetics; xanthine derivatives; cardiovascular preparations including calcium channel blockers and beta-blockers such as pindolol and antiarrhythmics; antihypertensives; diuretics; vasodilators including general coronary vasodilators; peripheral and cerebral vasodilators; central nervous system stimulants; cough and cold preparations, including decongestants; hypnotics; immunosuppressives; muscle relaxants; parasympatholytics; psychostimulants; sedatives; tranquilizers; naturally derived or genetically engineered lipoproteins; and restenoic reducing agents.

Other biologically active agents that can be used include alpha-interferon, genetically engineered epithelial cells, tacrolimus and dexamethasone.

A “prohealing” drug or agent, in the context of a blood-contacting implantable device, refers to a drug or agent that has the property that it promotes or enhances re-endothelialization of arterial lumen to promote healing of the vascular tissue. The portion(s) of an implantable device (e.g., a stent) containing a prohealing drug or agent can attract, bind and eventually become encapsulated by endothelial cells (e.g., endothelial progenitor cells). The attraction, binding, and encapsulation of the cells will reduce or prevent the formation of emboli or thrombi due to the loss of the mechanical properties that could occur if the stent was insufficiently encapsulated. The enhanced re-endothelialization can promote the endothelialization at a rate faster than the loss of mechanical properties of the stent.

The prohealing drug or agent can be dispersed in the body of the hygroscopic polymer substrate or scaffolding. The prohealing drug or agent can also be dispersed within a hygroscopic coating over a surface of an implantable device (e.g., a stent).

“Endothelial progenitor cells” refer to primitive cells made in the bone marrow that can enter the bloodstream and go to areas of blood vessel injury to help repair the damage. Endothelial progenitor cells circulate in adult human peripheral blood and are mobilized from bone marrow by cytokines, growth factors, and ischemic conditions. Vascular injury is repaired by both angiogenesis and vasculogenesis mechanisms. Circulating endothelial progenitor cells contribute to repair of injured blood vessels mainly via a vasculogenesis mechanism.

In some embodiments, the prohealing drug or agent can be an endothelial cell (EDC)-binding agent. In certain embodiments, the EDC-binding agent can be a protein, peptide or antibody, which can be, e.g., one of collagen type 1, a 23 peptide fragment known as single chain Fv fragment (scFv A5), a junction membrane protein vascular endothelial (VE)-cadherin, and combinations thereof. Collagen type 1, when bound to osteopontin, has been shown to promote adhesion of endothelial cells and modulate their viability by the down regulation of apoptotic pathways. S. M. Martin, et al., J. Biomed. Mater. Res., 70A:10-19 (2004). Endothelial cells can be selectively targeted (for the targeted delivery of immunoliposomes) using scFv A5. T. Volkel, et al., Biochimica et Biophysica Acta, 1663:158-166 (2004). Junction membrane protein vascular endothelial (VE)-cadherin has been shown to bind to endothelial cells and down regulate apoptosis of the endothelial cells. R. Spagnuolo, et al., Blood, 103:3005-3012 (2004).

In a particular embodiment, the EDC-binding agent can be the active fragment of osteopontin, (Asp-Val-Asp-Val-Pro-Asp-Gly-Asp-Ser-Leu-Ala-Try-Gly). Other EDC-binding agents include, but are not limited to, EPC (epithelial cell) antibodies, RGD peptide sequences, RGD mimetics, and combinations thereof.

In further embodiments, the prohealing drug or agent can be a substance or agent that attracts and binds endothelial progenitor cells. Representative substances or agents that attract and bind endothelial progenitor cells include antibodies such as CD-34, CD-133 and vegf type 2 receptor. An agent that attracts and binds endothelial progenitor cells can include a polymer having nitric oxide donor groups.

The foregoing biologically active agents are listed by way of example and are not meant to be limiting. Other biologically active agents that are currently available or that may be developed in the future are equally applicable.

In a more specific embodiment, optionally in combination with one or more other embodiments described herein, the implantable device of the invention comprises at least one biologically active agent selected from paclitaxel, docetaxel, estradiol, nitric oxide donors, super oxide dismutases, super oxide dismutase mimics, 4-amino-2,2,6,6-tetramethylpiperidine-1-oxyl (4-amino-TEMPO), tacrolimus, dexamethasone, dexamethsone-acetate, rapamycin, rapamycin derivatives, 40-O-(2-hydroxy)ethyl-rapamycin (everolimus), 40-O-(2-ethoxy)ethyl-rapamycin (biolimus), 40-O-(3-hydroxy)propyl-rapamycin, 40-O-[2-(2-hydroxy)ethoxy]ethyl-rapamycin, 40-O-tetrazole-rapamycin, 40-epi-(N1-tetrazolyl)-rapamycin (zotarolimus), pimecrolimus, imatinib mesylate, midostaurin, clobetasol, progenitor cell-capturing antibodies, prohealing drugs, cRGD, prodrugs thereof, co-drugs thereof, and a combination thereof. In a particular embodiment, the bioactive agent is everolimus. In another specific embodiment, the bioactive agent is clobetasol.

In some embodiments, optionally in combination with one or more other embodiments described herein, the at least one biologically active agent specifically cannot be one or more of any of the bioactive drugs or agents described herein.

### Coating Construct

According to some embodiments of the invention, optionally in combination with one or more other embodiments described herein, a coating disposed over a balloon for an implantable device (e.g., a stent) can be a multi-layer structure that includes at least one reservoir layer and a hygroscopic layer, which are layer (2) and layer (3) described below, and can include layer (1) as described below:

(1) a primer layer;

(2) a reservoir layer (also referred to “drug layer” or “drug matrix”), which can be a drug-polymer layer including at least one polymer (drug-polymer layer) or, alternatively, a polymer-free drug layer; and

(3) a hygroscopic layer.

The primer layer can be formed of any polymer suitable for an implantable device, which can be a bioabsorbable or biostable polymer. The reservoir layer or drug layer can include a bioactive agent with or without a hygroscopic polymer described herein.

In some embodiments, layer (2) and layer (3) can optionally include a bioabsorbable or biostable polymer.

### Method of Fabricating Implantable Device

Other embodiments of the invention, optionally in combination with one or more other embodiments described herein, are drawn to a method of fabricating an implantable device comprising a balloon. In one embodiment, the method comprises forming a hygroscopic layer or coating on the balloon.

Under the method, a portion of the implantable device or the whole device itself can be formed of the material containing a biodegradable or biostable polymer or copolymer. The method can deposit a coating having a hygroscopic layer wherein the coating has a range of thickness over a balloon for an implantable device. In certain embodiments, the method deposits over at least a portion of the implantable device a coating that has a thickness of ≦about 30 micron, or ≦about 20 micron, or ≦about 10 micron, or ≦about 5 micron.

Non-limiting examples of polymers, which may or may not be the hygroscopic polymers defined above, that can be used to fabricate an implantable device include poly(N-acetylglucosamine) (Chitin), Chitosan, poly(hydroxyvalerate), poly(lactide-co-glycolide), poly(hydroxybutyrate), poly(hydroxybutyrate-co-valerate), polyorthoester, polyanhydride, poly(glycolic acid), poly(glycolide), poly(L-lactic acid), poly(L-lactide), poly(D,L-lactic acid), poly(L-lactide-co-glycolide), poly(D,L-lactide), poly(caprolactone), poly(trimethylene carbonate), polyethylene amide, polyethylene acrylate, poly(glycolic acid-co-trimethylene carbonate), co-poly(ether-esters) (e.g., PEO/PLA), polyphosphazenes, biomolecules (e.g., fibrin, fibrinogen, cellulose, starch, collagen and hyaluronic acid), polyurethanes, silicones, polyesters, polyolefins, polyisobutylene and ethylene-alphaolefin copolymers, acrylic polymers and copolymers other than polyacrylates, vinyl halide polymers and copolymers (e.g., polyvinyl chloride), polyvinyl ethers (e.g., polyvinyl methyl ether), polyvinylidene halides (e.g., polyvinylidene chloride), polyacrylonitrile, polyvinyl ketones, polyvinyl aromatics (e.g., polystyrene), polyvinyl esters (e.g., polyvinyl acetate), acrylonitrile-styrene copolymers, ABS resins, polyamides (e.g., Nylon 66 and polycaprolactam), polycarbonates, polyoxymethylenes, polyimides, polyethers, polyurethanes, rayon, rayon-triacetate, cellulose and derivates thereof (e.g., cellulose acetate, cellulose butyrate, cellulose acetate butyrate, cellophane, cellulose nitrate, cellulose propionate, cellulose ethers, and carboxymethyl cellulose), and copolymers thereof.

Additional representative examples of polymers that may be suited for fabricating an implantable device include ethylene vinyl alcohol copolymer (commonly known by the generic name EVOH or by the trade name EVAL), poly(butyl methacrylate), poly(vinylidene fluoride-co-hexafluoropropylene) (e.g., SOLEF 21508, available from Solvay Solexis PVDF of Thorofare, N.J.), polyvinylidene fluoride (otherwise known as KYNAR, available from ATOFINA Chemicals of Philadelphia, Pa.), poly(tetrafluoroethylene-co-hexafluoropropylene-co-vinylidene fluoride), ethylene-vinyl acetate copolymers, and polyethylene glycol.

### Method of Treating or Preventing Disorders

An implantable device according to the present invention can be used to treat, prevent or diagnose various conditions or disorders. Examples of such conditions or disorders include, but are not limited to, atherosclerosis, thrombosis, restenosis, hemorrhage, vascular dissection, vascular perforation, vascular aneurysm, vulnerable plaque, chronic total occlusion, patent foramen ovale, claudication, anastomotic proliferation of vein and artificial grafts, arteriovenous anastamoses, bile duct obstruction, ureter obstruction and tumor obstruction. A portion of the implantable device or the whole device itself can be formed of the material, as described herein. For example, the material can be a coating disposed over at least a portion of the device.

In certain embodiments, optionally in combination with one or more other embodiments described herein, the inventive method treats, prevents or diagnoses a condition or disorder selected from atherosclerosis, thrombosis, restenosis, hemorrhage, vascular dissection, vascular perforation, vascular aneurysm, vulnerable plaque, chronic total occlusion, patent foramen ovale, claudication, anastomotic proliferation of vein and artificial grafts, arteriovenous anastamoses, bile duct obstruction, ureter obstruction and tumor obstruction. In a particular embodiment, the condition or disorder is atherosclerosis, thrombosis, restenosis or vulnerable plaque.

In one embodiment of the method, optionally in combination with one or more other embodiments described herein, the implantable device is formed of a material or includes a coating containing at least one ACAT inhibitor described above and optionally at least one biologically active agent selected from paclitaxel, docetaxel, estradiol, nitric oxide donors, super oxide dismutases, super oxide dismutase mimics, 4-amino-2,2,6,6-tetramethylpiperidine-1-oxyl (4-amino-TEMPO), tacrolimus, dexamethasone, dexamethasone-acetate, rapamycin, rapamycin derivatives, 40-O-(2-hydroxy)ethyl-rapamycin (everolimus), 40-O-(2-ethoxy)ethyl-rapamycin (biolimus), 40-O-(3-hydroxy)propyl-rapamycin, 40-O-[2-(2-hydroxy)ethoxy]ethyl-rapamycin, 40-O-tetrazole-rapamycin, 40-epi-(N1-tetrazolyl)-rapamycin (zotarolimus), pimecrolimus, imatinib mesylate, midostaurin, clobetasol, progenitor cell-capturing antibodies, prohealing drugs, cRGD, prodrugs thereof, co-drugs thereof, and a combination thereof.

In certain embodiments, optionally in combination with one or more other embodiments described herein, the implantable device used in the method is selected from stents, grafts, stent-grafts, catheters, leads and electrodes, clips, shunts, closure devices, valves, and particles. In a specific embodiment, the implantable device is a stent.

While particular embodiments of the present invention have been shown and described, it will be obvious to those skilled in the art that changes and modifications can be made without departing from this invention in its broader aspects. Therefore, the claims are to encompass within their scope all such changes and modifications as fall within the true sprit and scope of this invention.

1. An implantable device comprising a balloon, the balloon comprising a coating that comprises a hygroscopic layer over a drug layer that comprises a bioactive agent,
wherein the hygroscopic layer has a water absorbing capacity of between about 20 wt % and about 500 wt % by the dry weight of the hygroscopic layer within about 5 to 60 minutes upon exposure to an aqueous environment,
wherein the hygroscopic layer comprises a hygroscopic polymer; and
wherein the hygroscopic polymer is an amphiphilic polymer selected from the group consisting of polyethylene glycol- poly(caprolactone)-di- or tri-blocks (PEG-PCL) copolymers, polyethylene glycol-polylactide di- or tri-blocks (PEG-PLA) copolymers, polyethylene glycol-poly(lactide-glycolide) di-or tri-blocks (PEG-PLGA) copolymers, and PLURONIC® copolymers.

2. The implantable device of claim 2, wherein the bioactive agent is selected from the group consisting of paclitaxel, docetaxel, estradiol, 17-beta-estradiol, nitric oxide donors, super oxide dismutases, super oxide dismutases mimics, 4-amino-2,2,6,6-tetramethylpiperidine-1-oxyl (4-amino-TEMPO), biolimus, tacrolimus, dexamethasone, dexamethasone-acetate, rapamycin, rapamycin derivatives, 40-O-(2-hydroxy)ethyl-rapamycin (everolimus), 40-O-(3-hydroxy)propyl-rapamycin, 40-O-[2-(2-hydroxy)ethoxy]ethyl-rapamycin, and 40-O-tetrazole-rapamycin, 40-epi-(N1-tetrazolyl)-rapamycin (ABT-578), γ-hiridun, clobetasol, pimecrolimus, imatinib mesylate, midostaurin, cRGD, prodrugs thereof, co-drugs thereof, and combinations thereof.

3-6. (canceled)

7. The implantable device of claim 1, wherein the amphiphilic polymer is selected from the group consisting of polyethylene glycol- poly(caprolactone)-di- or tri-blocks (PEG-PCL) copolymers.

8. (canceled)

9. The implantable device of claim 1, wherein the drug layer comprises a hygroscopic polymer and wherein the hygroscopic polymer is an amphiphilic polymer.

10-12. (canceled)

13. The implantable device of claim 9, wherein the amphiphilic polymer is selected from the group consisting of polyethylene glycol- poly(caprolactone)-di- or tri-blocks (PEG-PCL) copolymers.

14. (canceled)

15. The implantable device of claim 1, wherein the coating is capable of releasing up to about 50% to about 100% of the bioactive agent in the drug layer within about 5 to 60 minutes upon exposure to the aqueous environment.

16. The implantable device of claim 1, wherein the hygroscopic layer is frangible such that upon expansion of the balloon the hygroscopic layer breaks so as to allow the bioactive agent in the drug layer to quickly release into the aqueous environment.

17-20. (canceled)

21. A method of fabricating a coating including two or more bioactive agents on a balloon of an implantable device, the method comprising
forming a reservoir layer comprising a bioactive agent over the balloon,
forming a hygroscopic layer comprising a hygroscopic polymer over the reservoir layer, and
exposing the hygroscopic layer to a solution comprising one or more bioactive agent and water to cause the hygroscopic layer to uptake the solution,
wherein the hygroscopic layer has a water absorbing capacity of between about 20 wt % and about 500 wt % by the dry weight of the hygroscopic layer within about 5 to 60 minutes upon exposure to an aqueous environment; and
wherein the hygroscopic layer comprises an amphiphilic polymer selected from the group consisting of polyethylene glycol- poly(caprolactone)-di- or tri-blocks (PEG-PCL) copolymers, polyethylene glycol-polylactide di- or tri-blocks (PEG-PLA) copolymers, polyethylene glycol-poly(lactide-glycolide) di-or tri-blocks (PEG-PLGA) copolymers, and PLURONIC® copolymers.

22. The method of claim 21, wherein the reservoir layer comprises a polymer.

23. The method of claim 21, wherein the bioactive agent in the reservoir layer and the one or more bioactive agent in the solution are the same or different.

24. The method of claim 21, wherein the bioactive agent in the reservoir layer and the one or more bioactive agent in the solution are independently selected from the group consisting of paclitaxel, docetaxel, estradiol, 17-beta-estradiol, nitric oxide donors, super oxide dismutases, super oxide dismutases mimics, 4-amino-2,2,6,6-tetramethylpiperidine-1-oxyl (4-amino-TEMPO), biolimus, tacrolimus, dexamethasone, dexamethasone-acetate, rapamycin, rapamycin derivatives, 40-O-(2-hydroxy)ethyl-rapamycin (everolimus), 40-O-(3-hydroxy)propyl-rapamycin, 40-O-[2-(2-hydroxy)ethoxy]ethyl-rapamycin, and 40-O-tetrazole-rapamycin, 40-epi-(N1-tetrazolyl)-rapamycin (ABT-578), γ-hiridun, clobetasol, pimecrolimus, imatinib mesylate, midostaurin, cRGD, prodrugs thereof, co-drugs thereof, and combinations thereof.

25. A method of treating, preventing, or ameliorating a medical condition, comprising implanting in a patient an implantable device comprising a balloon according to claim 1, wherein the medical condition is selected from the group consisting of restenosis, atherosclerosis, thrombosis, hemorrhage, vascular dissection or perforation, vascular aneurysm, vulnerable plaque, chronic total occlusion, claudication, anastomotic proliferation, bile duct obstruction, ureter obstruction, tumor obstruction, or combinations of these.

26. An implantable device comprising a balloon, the balloon comprising a coating that comprises a hygroscopic layer over a drug layer, the drug layer comprising a bioactive agent,
wherein the hygroscopic layer has a water absorbing capacity of between about 20 wt % and about 500 wt % by the dry weight of the hygroscopic layer within about 5 to 60 minutes upon exposure to an aqueous environment;
wherein the hygroscopic layer comprises a hygroscopic polymer;
wherein the hygroscopic layer is free of non-hygroscopic polymers; and
wherein the hygroscopic polymer is an amphiphilic polymer selected from the group consisting of polyethylene glycol- poly(caprolactone)-di- or tri-blocks (PEG-PCL) copolymers, polyethylene glycol-polylactide di- or tri-blocks (PEG-PLA) copolymers, polyethylene glycol-poly(lactide-glycolide) di-or tri-blocks (PEG-PLGA) copolymers, and PLURONIC® copolymers.

27. The implantable device of claim 1, wherein the amphiphilic polymer is selected from the group consisting of polyethylene glycol-polylactide di- or tri-blocks (PEG-PLA) copolymers.

28. The implantable device of claim 1, wherein the amphiphilic polymer is selected from the group consisting of polyethylene glycol-poly(lactide-glycolide) di-or tri-blocks (PEG-PLGA) copolymers.

29. The implantable device of claim 1, wherein the amphiphilic polymer is selected from the group consisting of PLURONIC® copolymers.
